(19) United States
(12) Patent Application Publication
(10) Pub. No.: US 2026/0283714 A1
(43) Pub. Date: Sep. 24, 2026

(54) MEDICAL ROBOT GUIDANCE SYSTEM HAVING AN INTEGRATED TOUCH DISPLAY, AND OPERATING METHOD

(71) Applicant: B. Braun New Ventures GmbH, Freiburg im Breisgau (DE)

(72) Inventors: Sebastian Zepf, Wurmlingen (DE); Ann-Kathrin Huber, Moenchweiler (DE); Alisa Post, Immenstadt (DE); Amir Sarvestani, Freiburg (DE)

(21) Appl. No.: 19/100,637

(22) PCT Filed: Aug. 3, 2023
(86) PCT No.: PCT/EP2023/071615

§ 371 Date: Feb. 3, 2025

(30) Foreign Application Priority Data
Aug. 4, 2022 (DE) 10 2022 119 613.5

(51) Int. Cl. A61B 34/00; A61B 34/35; A61B 90/00; A61B 90/20; G06F 3/0482; G06F 3/0488

(52) U.S. Cl. CPC A61B 34/25; A61B 34/35; A61B 34/74; A61B 90/20; A61B 90/361; G06F 3/0482; G06F 3/0488

(57) ABSTRACT

A medical robot guiding system for a surgical intervention on a patient includes a robot with a movable robot arm, a robot head terminally connected to the robot arm, an end effector on the robot head or as a robot head, a control unit to control and move at least the robot, and at least one touch display adapted to visually output at least one operating menu and to detect a touch-sensitive input as operating input and to send it to the control unit in order to control the robot. The at least one touch display is rigidly fixed to the robot head and moves along with it. The system can be operated using a robot operating method and a computer-readable storage medium.

### CROSS-REFERENCE TO RELATED APPLICATIONS

This application is the United States national phase entry of International Application No. PCT/EP 2023/071615, filed on Aug. 3, 2023, and claims priority to German Application No. 10 2022 119 613.5, filed on Aug. 4, 2022. The contents of International Application No. PCT/EP 2023/071615 and German Application No. 10 2022 119 613.5 are incorporated by reference herein in their entireties.

### FIELD

The present disclosure relates to a medical, in particular surgical, robot guiding system for a medical procedure, in particular a surgical intervention, on a patient. For this purpose, the robot guiding system comprises a robot having a robot arm movably connected to a robot base and a robot head terminally connected to the robot arm. In particular, the robot head has an end effector or an end effector itself forms the robot head. Furthermore, the robot guiding system has a (central) control unit (in particular with a processor and a memory unit) which is adapted to control and move at least the robot, in particular the robot arm and the robot head (and thus in particular the end effector). Furthermore, the robot guiding system comprises at least one touch display/touchscreen/touch-sensitive screen which is adapted to visually output at least one operating menu (for controlling) and to detect a touch-sensitive input as operating input and to send it to the control unit in order to control the robot in particular. In addition, the disclosure relates to a (robot) operating method and a computer-readable storage medium.

### BACKGROUND

Surgical guiding systems are more and more often used for interventions, in particular minimally invasive interventions. The number of functions of medical systems is increasing rapidly due to technological development and the increasing specialization of various (sub-)systems with corresponding integration.

The progressive expansion of the guiding systems to include this multitude of different functionalities makes it increasingly difficult to provide the user with a central operating function or central operating modality that allows them to maintain an overview of the functions and control.

For the operation of a robotic guiding system, touch displays are currently provided in the state of the art, for example on a medical trolley, a medical tower or on a base of a medical (operating) microscope, via which a medical professional can provide (operating) input for a corresponding control via guidance through an (operation/operating) menu.

However, one problem is that the touch display is not arranged centrally in the area of the intervention, but away from it. This makes it difficult to reach the touch display on the one hand and to meet hygiene requirements on the other hand, since the touch display is not provided to be sterile. Due to the distance on the one hand and the sterility requirement on the other hand, surgeons conducting the intervention are usually unable to operate the touch display themselves.

US 2005/0041282 A1, for example, discloses a surgical microscope as a robot guiding system with a touch display that is rigidly attached to a base/trolley of the surgical microscope. The touch-sensitive display can be used to control different functions via different areas of the display. Due to this configuration, however, the surgeon has to instruct another medical professional to control a function so that this professional then executes the control.

### SUMMARY

It is therefore the object of the present disclosure to avoid or at least reduce the disadvantages of the prior art and in particular to provide a medical robot guiding system and an operating method which provides a user, such as a medical professional, in particular a surgeon, with an intuitive and clearly arranged operation control with an intuitive user interface which, during a medical procedure such as an intervention, enables them to have full control over the system or several systems used. In particular, the operation control is intended to bring together different operating modalities centrally and preferably to be provided in an area that is easily accessible to the surgeon.

Thus, a basic idea of the present disclosure is to provide an operating modality, such as a control of the robot, in the area of a robot head or of an end effector. In contrast to the prior art, the touch display as input unit and output unit is not provided at a separate, remote location, such as on a medical tower or the like, but directly on a movable part of the robot, i.e. on the terminal robot head and thus in the area of the end effector.

In other words, the at least one touch display is rigidly connected/attached/fixed to the robot head, in particular to the end effector, and moves with it. Instead of providing a static touch display on the robot base, for example on a carriage of a robot-assisted surgical microscope, according to the present disclosure, the touch display on the robot head is moved dynamically. Since the end effector is arranged on the robot head or the end effector itself forms the robot head, the operating modality in the form of the touch display is arranged directly in the area of the end effector and can be reached easily and safely by a surgeon. The surgeon may then use the dynamically moving touch display to control various functions of the operating modalities centrally, so to speak, or they are provided with the option of controlling at least one movement of the robot and thus of the robot head centrally in the area of the end effector. In particular, the surgeon may control different functions via the touch display, for example by displaying an associated operating menu for the corresponding function.

In yet other words, the disclosure describes a touch display/touch screen/touch-sensitive screen embedded in a robot-assisted guiding system/robot guiding system for medical interventions. The touch display is connected by data technology to a central control unit (as an execution unit for software) and enables flexible control of various functions, for example in non-sterile and sterile environments, as well as visualization of information. The robot guiding system may, for example, include both visualization-based guidance (e.g. a surgical microscope) and guidance of instruments (e.g. a trocar).

In yet other words, an embedded touch display on or at a robotic end effector (on or as a robot head) is proposed herein, which enables a user to flexibly provide information and offer various control options in both sterile and non-sterile environments (e.g. via an external monitor). The touch display is rigidly connected to the robot end effector and in particular is oriented in such a way that the surgeon has a good viewing angle in the most common surgical positions (in particular poses) of the guiding system. The touch display may show both interactive content (such as the operating menu) and non-interactive content (such as an annotation). The visualized content may, for example, display the same content as a larger main monitor of the surgeon (not arranged on the robot arm) or as other control displays that are not attached to the control device. Alternatively or additionally, independent content, which may be situation-dependent, may preferably also be displayed by the touch display.

The term “position” refers to a geometric position in three-dimensional space, which in particular is specified using coordinates of a Cartesian coordinate system. In particular, the position can be specified by the three coordinates X, Y and Z.

The term “orientation” in turn indicates an alignment (for example at the position) in space. It can also be said that the orientation indicates an alignment with an indication of direction or rotation in three-dimensional space. In particular, the orientation can be specified using three angles.

The term “pose” covers both a position and an orientation. In particular, the pose can be specified using six coordinates, three position coordinates X, Y and Z and three angular coordinates for the orientation.

An operating input may, for example, be a control command associated with a selection of the operating menu, which is sent to the control unit so that it executes the corresponding function directly or indirectly via a control unit of a subsystem, for example. For example, a central control unit may control a visualization system indirectly via a sub-control unit thereof, or it may control a navigation system indirectly via a sub-control unit thereof and have it set a waypoint, for example.

Advantageous embodiments are explained in particular below.

According to one embodiment, the robot guiding system may be configured in the form of a surgical (surgery) microscope with a microscope head connected to the robot arm as a robot head or in the form of a navigation system with a camera system connected to the robot arm, in particular with a laser system. The microscope head may be actively controlled and moved and a surgeon can make operating inputs directly on the microscope head via the touch display, for example with regard to a zoom, an alignment and/or an illumination and/or a movement/displacement of the microscope head (pose change). In the case of a navigation system, for example, the view of the touch display and/or the view on the navigation monitor can be changed and adapted in order to obtain a better overview or to be able to track instruments even better.

In particular, the robot arm of the robot may be configured in such a way that the robot head is adjustable both in its position and in its orientation, i.e. in its pose (or has six degrees of freedom/6DOF). In particular, the robot arm may have at least a first and a second robot arm segment, which are connected to each other via a joint, and the robot head may be connected to the robot arm via a further joint and the robot arm may be connected to the robot base via an additional joint. In particular, the joint of the first and second robot arm segments may have a degree of rotational freedom for rotation about an axis of rotation, wherein this axis of rotation may be arranged in a kinematic position of the robot arm in particular in a horizontal direction (i.e. perpendicular to an up-down direction) in order to provide a kind of cantilever (similar to an excavator arm). In particular, the robot may be configured in the form of an articulated arm robot.

In particular, the robot head is rigidly configured and connected to the robot arm via a bearing or joint.

Preferably, the robot arm may have at least three robot arm segments, each of which is connected to each other via a joint. In particular, the robot (or the robot's control unit) may control the position and orientation of the robot head via a multi-link actuated kinematic system.

Preferably, the surgical microscope is a digital microscope that creates a digital microscopic image via a sensor, such as a CMOS sensor, and provides it digitally.

Preferably, the touch display may be arranged on a lateral side of the robot head (i.e. not on a rear side in extension of a longitudinal axis of the robot head). In particular, the touch display may be arranged on the robot head in such a way that a normal to the display surface is substantially perpendicular to a longitudinal axis of the robot head, such as a viewing axis of a surgical microscope.

In particular, the touch display may be arranged laterally (i.e. not on a rear side) in an embodiment of a microscope head as a robot head on an area opposite the optical output. Alternatively or additionally, the touch display in a microscope head may be arranged on a side facing away from the optical output, i.e. quasi opposite (one front side is adapted for the optical output, in particular has a lens of an optical system, the opposite front side has the touch display). It can also be said that the at least one touch display is arranged “on top” of the microscope (camera) head on a lateral and/or upper side, while the optical output is provided “below”. An optional operating button or an actuation button or an input means in the form of a joystick or a 3D mouse may be arranged at an axial position between the optical output (bottom) and the touch display (top) as viewed in the longitudinal axis direction of the robot head, in particular directly below the touch display. Preferably, the robot arm may also be connected to the robot head via a joint between the optical output (bottom) and the touch display (top) as seen in the longitudinal axis direction of the robot head. In particular, the touch display represents the “uppermost” or most terminal element or component which is provided laterally and/or frontally on the robot head.

Preferably, the touch display may have a round outer contour or shape, in particular a circular outer contour or shape.

According to an embodiment, the robot head in the form of a microscope head may have a cylinder-shaped/cylindrical base body, wherein one round front side forms the optical output for the digital microscope or for the microscope camera and the opposite round front side has the circular touch display.

In particular, an additional input means, for example in the form of a joystick or a 3D mouse, may be provided in extension or at the same height opposite the connection of the robot arm. If the robot head is connected to the robot arm on one lateral side via a joint, in particular a pivot joint, the input means is provided on the opposite lateral side, starting from the connection in extension of an axis perpendicular to an optical axis (it can also be said perpendicular to a longitudinal axis of the robot head).

In particular, the robot guiding system may be adapted to display a menu structure for accessing various functionalities via the touch display. Exemplary applications of the touch display are listed in particular below. The touch display may therefore not only display a single operating menu or a single indicator, but may also provide the surgeon with a large number of operating menus to control various functions. In particular, it is possible to switch from a top menu structure to several submenu structures and back.

Preferably, the robot guiding system may be adapted to output a robot control menu as an operating menu via the touch display in order to control the robot via an operating input (a movement), in particular in order to control a robotic movement in six degrees of freedom, for example in six translational directions (in each case two opposite directions of a Cartesian coordinate system +X/−X, +Y/−Y, +Z/−Z) and/or six rotational directions (clockwise or counterclockwise rotations about the respective axis). In other words, the touch display may be adapted to display an (operating) menu structure for accessing the function adapted to control robotic movements in particular six degrees of freedom.

In particular, the robot guiding system may be adapted to output a visualization control menu as an operating menu via the touch display in order to control/change settings of a visualization system, in particular a zoom, a focus and/or an illumination intensity (as settings). In other words, the touch display may be adapted to display a menu structure for accessing the function: setting settings of a visualization system (such as a surgical microscope) such as zoom, focus and light intensity via in particular a touch bar/slider.

According to a further embodiment, the robot guiding system may be adapted to switch between at least two different control menus, in particular between at least a robot control menu and a visualization control menu, in order to control at least two different functions of the robot guiding system, in particular a movement of the robot as a first function and visualizing as a second function.

In particular, the robot guiding system may be adapted to output a visualization control menu as an operating menu via the touch display, which allows a control of different light/imaging modes of the visualization system, in particular a control of a fluorescence for in particular ICG (indocyanine green), 5-ALA (5-aminolevulinic acid).

In particular, the robot guiding system may also be adapted to output a navigation control menu as an operating menu via the touch display, which allows waypoints and/or robot configurations and/or navigation positions to be stored in relation to the patient (or a patient position) and in particular provides a so-called “hold and drive” function, in which a long press on one of the stored and displayed data causes moving to the stored points or predefined positions.

In particular, the robot guiding system may also be adapted to output a navigation control menu as an operating menu via the touch display, which allows or provides control of navigation sequences, in particular point digitization and verification for patient registration and calibration/activation of tools.

In particular, the robot guiding system may also be adapted to output an instrument control menu as an operating menu via the touch display, which allows control of instrument guidance functions such as moving the robot or the instrument as an end effector on a target trajectory or switching an instrument function on or off.

In particular, the robot guiding system may further be adapted to output a media control menu as an operating menu via the touch display, which allows control of the recording of images (for example as current snapshots) and/or video data of a visualization system. This allows the surgeon to create an image at an initial point in time using an operating input, for example, and to have this image output on the touch display or an external monitor at a later point in time, for example to make a before/after comparison or to call up information on the intervention site.

In particular, the robot guiding system may further be adapted to output a media control menu as an operating menu via the touch display, which allows control of the playback and management of the recorded media data, wherein the played video may in particular be shown on other displays (in addition or alternatively to the touch display).

In particular, the robot guiding system may further be adapted to output a navigation control menu as an operating menu via the touch display, which allows or provides a control of settings for the information displayed on the visualization monitor, in particular to show and hide planned trajectories, operation targets or other navigation information.

In particular, the robot guiding system may also be adapted to output a navigation control menu as an operating menu via the touch display, which provides a control for switching between different monitor layouts of the main visualization monitor.

In particular, additional situation-dependent content based on information from the control unit/control device may be displayed to save time for the user when navigating through the operating menu. In particular, the following situation-dependent content (e.g. depending on a robot configuration or a current status of an operation plan) may be displayed, individually or in a selectable combination with each other:
settings of a visualization system may be highlighted after the guiding system has been repositioned to allow quick adjustment of the visualization parameters to the new position;
if a movement of a navigated instrument is detected, the settings of the instrument guidance may be highlighted;
visualizing a current state/current function of multi-purpose hardware buttons located in the vicinity of the display;
representation of a safety stop button on the touch display for interrupting automated robotic movements;
displaying notifications to the user (such as information, warnings and errors) both with and without prompting for user feedback;
visualizing a distance to the target for instrument guidance with depth tracking/depth indication;
rendering of an image of a visualization system for (rough) positioning of the system;
in particular, an indicator mode of the touch display may be switched to a so-called “trackpad” mode, in which:
   a mouse icon is activated on the main visualization screen, in particular a surgical monitor, allowing the user to operate the visualization screen as a computer screen with a mouse/laptop trackpad (via the touch display); or
   it allows the user to scroll through the navigation views/slices (segmentations) on the main visualization screen, in particular on the OR monitor.

In particular, the touch display fixed to the robot head may have a sterile sheathing, which is preferably designed to be exchangeable in order to provide a sterile barrier with respect to a sterile intervention site. For example, for operating in a sterile environment, the touch display may be covered with a medical (surgical) drape with transparency, wherein in particular connection points/coupling points for the surgical drape are provided.

According to a further embodiment, an inertial measurement unit (IMU) may further be provided on or in the robot head, in particular may be provided on or in the touch display or is attached to the end effector, in order to detect a position and/or orientation of the touch display or of the robot head, and the robot guiding system, in particular the control unit, may be adapted to adapt an alignment of a visual output of the touch display based on the detected position and/or orientation, in particular to adapt an output (such as of the operating menu) so that it is always displayed in a constant horizontal alignment. In other words, an inertial measurement unit (IMU) may optionally be connected to the touch display or to the robot head or to the end effector in order to be able to change the alignment of the visualized content in different positions and/or orientations, in particular extreme positions, of the guiding system. Alternatively or additionally, a position and/or orientation of the touch display or of the robot head may preferably be detected via robot kinematics or a real-time configuration of the robot with its robot arm segments and its robot head as end effector or with connected end effector. Furthermore, a position and/or orientation of the touch display or of the robot head may preferably be detected via the navigation camera of the navigation system and corresponding data processing. In particular, the control unit may be adapted to adapt an indicator on the basis of the detected pose of the touch display so that it is optimally displayed relative to a predetermined position in space, i.e. the position at which the head of an surgeon is located (for example, this position may be detected by a navigation camera). In particular, this may include a rotation of the view of the touch display so that the view is preferably displayed approximately horizontally relative to a floor of an operating room, and thus relative to the surgeon, as well as an elongation or compression of the view in order to provide a view that is as neutral and natural as possible in the detected position of the surgeon (similar to arrows or labels on a roadway or a lane marking, which are adapted (elongated) for a driver so that the driver may recognize the information as well as possible). In particular, the control unit or the touch display may be adapted to output such a projection on a virtual surface via the touch display, wherein the surface is perpendicular to a line of sight between the surgeon and the touch display, so that the surgeon does not see a distorted representation (when the touch display is at an angle to a line of sight), but such a representation similar to when they would look perpendicularly at the touch display.

Preferably, the touch display may have a radio communication module in order to (independently) establish a wireless data connection to the control unit, in particular via WLAN or Bluetooth. According to an alternative embodiment, the touch display may also be connected to the control unit via a data cable. In other words, to control various functionalities of the guiding system for the situation-dependent representation of content, the touch display may be connected to the control unit (as a computer system) via a touch display control device by cable or wirelessly (with a radio communication module). In particular, the touch display itself has an independent sub-control unit as a computer system in order to form an independent control component which may be integrated into the overall system and may be coupled to the (central) control unit using data technology.

Further preferably, the touch display may have a display diagonal or a display diameter of at least 4 cm and/or a maximum of 20 cm. In other words, the size of the touch display may be between 4 cm and 20 cm. In particular, the touch display may have a square, rectangular or round form factor. This size and shape may, for example, be based on the number of functions and the size of the connected robot effector. The size and shape help to integrate the touch display advantageously into the area of the end effector.

According to an embodiment, the robot head may have at least one (physical) actuation button (hardware button), preferably three actuation buttons, and the touch display may be arranged directly adjacent to the button and may be adapted to display the current function status (/the current mode of operation) of the button. This creates a multi-purpose button or a multi-function button that may detect different operating inputs for different operating menus. This may also increase safety in particular if, for example, an input via the touch display fails or an input cannot be made accurately due to dirty surgical gloves.

In particular, the touch display has a color display to show colored operating menus and/or information.

In a further embodiment, in addition to the touch display on the robot head, a touch display may be provided on the robot arm and/or on a robot base.

In particular, the robot guiding system may be configured as a robot guiding unit and all components may be integrated in this unit or in a single module. In particular, the robot guiding system may be configured to be mobile and may be provided and moved autonomously in an operating room, for example configured as a mobile surgical microscope on whose microscope head (as robot head and end effector) the touch display is arranged.

The objects are solved with respect to a medical robot control method, in particular for a robot guiding system according to the present disclosure, by the steps of: outputting at least one visual operating menu via a touch display that is rigidly attached to a robot head of a robot; detecting a touch-sensitive input as an operating input via the touch display; sending the operating input to a control unit which is adapted to control at least the robot; controlling, based on the operating input, a function, in particular a movement of a robot, by the control unit. By this step, as described above, a flexible and central operating method may be provided, via which the surgeon can perform different functions, for example a movement of a robot or a setting of a visualization system.

Preferably, the (operating) method may further comprise the steps of:
detecting a (change in) position and orientation of the robot head, in particular by an inertial measurement unit (IMU) or by detected robot kinematics or by a navigation camera of a navigation system; sending the detected position and orientation to the control unit; calculating an alignment of the visual operating menu adapted to the position and orientation; and outputting the adapted visual representation of the operating menu. In this way, the surgeons can be provided with the best possible or best-adapted visual output and, in particular, do not have to turn their head to read a label or information or reposition themselves in order to easily recognize the information of the representation, such as a text, when looking at the touch display from an angle.

With respect to a computer-readable storage medium, the objects are solved by comprising instructions which, when executed by a computer, cause the computer to execute the method steps of the operating method according to the present disclosure. In particular, the control unit of the robot guiding system may comprise such a computer-readable storage medium.

Any disclosure relating to the robot guiding system according to the present disclosure also applies to the operating method of the present disclosure, as well as vice versa.

### BRIEF DESCRIPTION OF THE DRAWINGS

The present invention is explained in more detail below with reference to the accompanying Figures, with reference to preferred embodiments.

FIG. 1 shows a schematic front view of a robot guiding system according to a preferred embodiment of the present disclosure;

FIG. 2 shows a schematic view of a functional relationship between the touch display, control unit and IMU of a robot guiding system according to a further preferred embodiment;

FIGS. 3a and 3b show an exemplary view of an operating menu in a tabular arrangement and in a circular arrangement;

FIG. 4 shows a schematic view of an exemplary submenu of a visualization control menu for setting a light intensity;

FIG. 5 shows a schematic view of an exemplary submenu of a media control menu for management and a playback function of a recorded video;

FIG. 6 shows a schematic view of an operating menu with only one stop button as interactive input;

FIG. 7 shows a schematic front view of a robot head or end effector with three buttons and a touch display;

FIG. 8 shows a schematic front view of a robot guiding system according to a further preferred embodiment of the present disclosure;

FIGS. 9 to 11 show different views of a robot guiding system comprising a robot head with a touch display arranged on a side facing away from the optical output, at the top of the microscope head; and

FIG. 12 shows a flowchart of an operating procedure according to a preferred embodiment.

The Figures are merely schematic in nature and are only intended to aid understanding of the invention. Identical elements are marked with the same reference signs. The features of the various embodiments can be interchanged.

### DETAILED DESCRIPTION

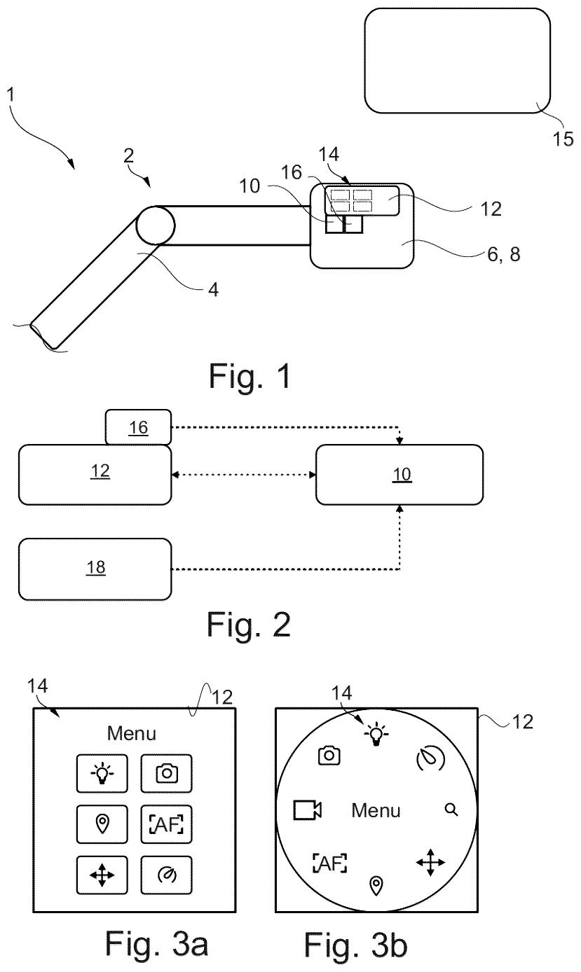

FIG. 1 shows a schematic front view of a medical robot guiding system 1 (hereinafter only referred to as guiding system) for a surgical intervention on a patient P. The guiding system 1 comprises a robot 2 with a movable robot arm 4 and a robot head 6 terminally connected to the robot arm 4. In this embodiment, the guiding system 1 is designed in the form of a robot-guided surgical microscope. The microscope head is provided as the robot head on the robot arm 4 with several robot arm segments. Therefore, the robot head or microscope head as a whole may also be referred to as the end effector 8 of the surgical microscope.

In order to control the robot 2 and its robot arm 4 and to set the microscope head as end effector 8 in a position and orientation, the guiding system 1 has a control unit 10. The control unit 10 may be designed as a central control unit, for example as a computer system that may process and control different functions, or it may have a subsystem of a robot control unit in order to control the robot accordingly.

Furthermore, the guiding system 1 has a touch display 12 as input and output unit. This is adapted to visually output different operating menus 14 and indicators for different functions, and to detect a touch-sensitive input as an operating input and send it to the control unit 10 in order to both control the robot 2 and to make settings on the visualization system.

In contrast to the prior art, however, the touch display for control is not attached to a static base of the guiding system 1, but the touch display 12 is rigidly fixed directly to the robot head 6 or to the end effector 8 and moves with it.

In this way, the touch display 12 moves in the area of the intervention and the surgeon has central visualizing of different operating menus 14 and can flexibly control the different functions of different subsystems via a single touch display. In this embodiment, the surgeon can select from at least one robot control menu and visualizing control menu or may switch between the individual control menus as operating menus 14. Using the robot control menu, the surgeon may, for example, effect instrument guidance or may move the robot head 6 so that the end effector 8 is in a better pose for the intervention. In the present case, the microscope head may be moved manually or automatically as an end effector via the operating input of the touch display 12 into a pose that allows a better view.

Further images may be output via an external (operating room) monitor 15, wherein a media output may be controlled by a further media control menu via the touch display 12. For example, a real-time image of the microscope may be output via the surgical monitor 15, wherein a brightness or a color contrast may be set via a media control menu.

In the robot guiding system 1 according to the first embodiment, the surgeon is thus provided with at least the three control menus: robot control menu, visualization control menu and media control menu, which may be operated centrally in order to control various functions flexibly and safely.

FIG. 2 shows a schematic functional view of an interaction between individual modules of a guiding system 1 according to a further, second preferred embodiment. Here, an inertial measurement unit (IMU) 16 is arranged directly on the touch display 12 in order to detect a movement (or a change in a movement via an acceleration) and to determine a new position and orientation of the touch display 12 after a movement of the robot on the basis of an initial pose.

The data from the IMU is sent directly to a central control unit 12. In addition, the central control unit 10 receives data from an end effector control unit 18 (as a sub-control unit). Furthermore, the central control unit 12 is in a two-way data connection with a touch display control unit (as a sub-control unit). This central control unit 12 then calculates a visual output on the basis of this data, which is then output on the touch display 12.

In particular, a view that is most favorable for the surgeon can be calculated in this way and can be output accordingly. Since, in contrast to the prior art, the touch display is no longer arranged statically, but moves dynamically with the end effector 8, the indicator changes relative to the surgeon in an alignment. If, for example, the robot arm 4 in FIG. 1 is swiveled upwards by 90°, the visual view of the touch display 12 would also be rotated by 90° counterclockwise relative to the surgeon without correction.

However, in order to allow the surgeon to continue to read the visual indicator easily, the control unit 12 calculates such an alignment, in the above example a rotation of the indicator relative to the touch display by 90° in a clockwise direction, so that the rotation and counter-rotation cancel each other out and allow intuitive reading by the surgeon. In other words, during a movement of the robot 2, the control unit calculates an alignment of the indicator that appears as constant as possible to the surgeon and, to a certain extent, enables a constant relation of the indicator of the touch display relative to the surgeon.

In one embodiment, for example, when changing from a front view on the touch display 12 (i.e. vertical view) to an oblique view, the visual output may also be displayed distorted accordingly (similar to a road marking in the form of lettering, which is also displayed elongated in order to provide the driver with the best possible view).

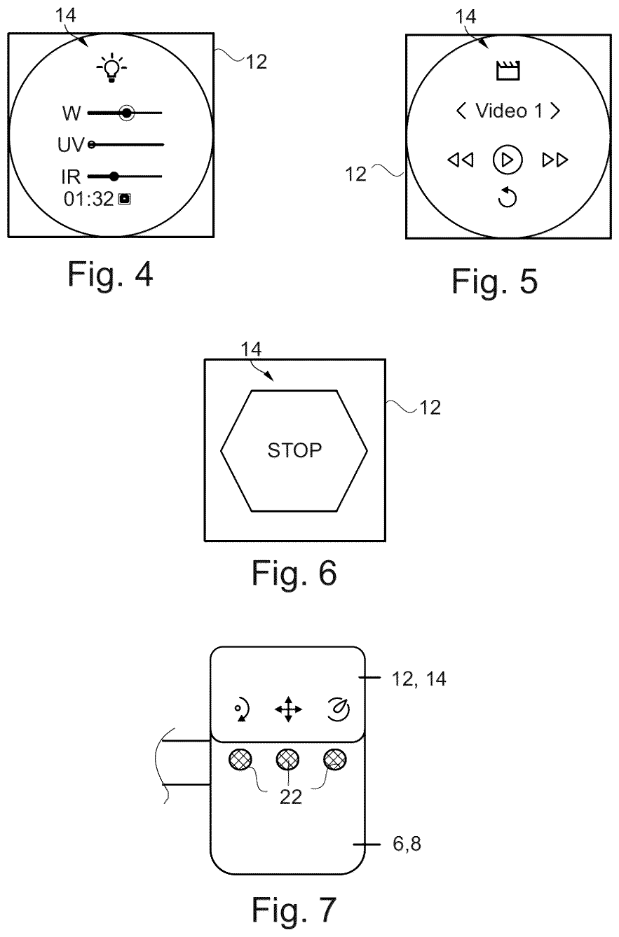

FIG. 3a and FIG. 3b are exemplary views of an operating menu represented by the touch display, wherein a table-shaped arrangement with rectangular, symbol-labeled (touch) buttons is shown in FIG. 3a and a circular arrangement of symbol-labeled (touch) buttons is shown in FIG. 3b. The user may use these to select a sub-menu, for example, which is shown in FIGS. 4 and 5 and explained below.

FIG. 4 shows a sub-menu of a lighting control menu when the symbol-labeled light bulb is selected in the main menu. In this embodiment, the user may set a brightness of a white light, as well as an intensity of a UV radiation and an IR radiation.

If the video symbol is selected by the user in the main menu in FIG. 3a or 3b, the menu jumps to the sub-menu of the video function shown in FIG. 5, in which the management of the video data and playback functions may be selected. The video may be played back on an external operating room monitor (not shown here).

FIG. 6 shows a schematic view of a (digital) stop button on the touch display 12 to stop the movement of the robot 2 or of the robot arm 4 during a movement (a type of emergency button). FIG. 7 shows a front view of an end effector of a robot guiding system 1 of another preferred embodiment. In this embodiment, three physical pushbuttons/actuation buttons 22 are provided on the end effector 8 on a straight line, equally spaced from each other. Parallel to the linear arrangement of the three actuation buttons 22 and above them, a touch display 12 is attached to the end effector, which displays the function status above the corresponding actuation button 22. In this way, several functions may be assigned to the three actuation buttons 22 and can be visualized accordingly by the touch display.

For example, the control unit 12 may assign different functions to the actuation buttons for different steps in the operation plan and can output them via the touch display.

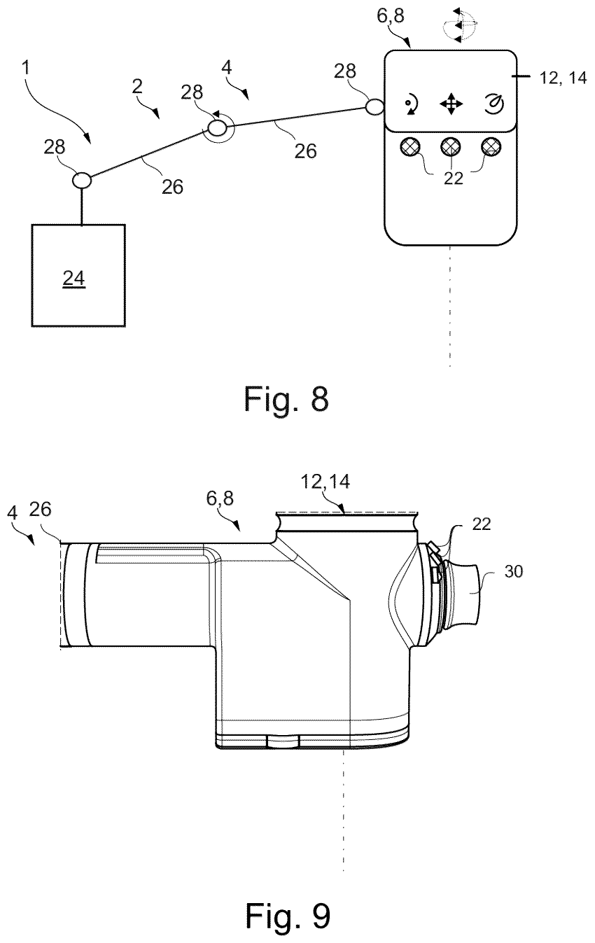

FIG. 8 shows a schematic front view of another embodiment of a guiding system 1. The robot 2 has a stationary robot base 24, to which the robot arm 4 is movably connected. The robot head 6 in the form of a (digital) microscope head is connected to the end of the robot arm 4 as an end effector 8, so that the robot arm 4 may adjust both the position and the orientation (i.e. the spatial pose) of the microscope head in order to assume a suitable image pose and to create a digital microscopic image. The (vertical) dashed line shows the optical axis of the microscope head, which passes through the optical system (not shown) and in extension a CMOS sensor for a digital image. By setting both the position (three degrees of freedom) and the orientation (three degrees of freedom), six degrees of freedom/6DOF (degree of freedom) may be set (within the robot kinematics).

In this embodiment, the robot arm 4 has several robot arm segments 26, each of which is connected to each other via a joint 28. The robot head 6 with the optical system is also connected to the robot arm 4 via a further joint 28 and the robot arm 4 is connected to the robot base 24 via a joint 28. In particular, the joint of the first and second robot arm segments 26 may have a degree of rotational freedom for rotation about an axis of rotation, wherein this axis of rotation may be arranged in particular in a horizontal direction (i.e. perpendicular to an up-down direction) in a kinematic position of the robot arm 4 shown in FIG. 8, in order to provide a kind of cantilever.

The robot head 6 is rigidly configured (or has a rigid robot head housing in which the optical system, the downstream CMOS sensor and any other electronics are housed). The robot 2 (or the control unit of the robot) may therefore control the position and orientation of the robot head 6 and adjust the optical axis accordingly via a multi-link actuated kinematic system.

In this embodiment, the touch display 12 is arranged on a lateral side of the robot head 6 (i.e. not on a rear side in extension of a longitudinal axis of the robot head). In particular, the touch display may be arranged on the robot head in such a way that a normal to the display surface is substantially perpendicular to a longitudinal axis of the robot head, such as a viewing axis of a surgical microscope.

The touch display 12 is arranged so that it is at the top of the robot head 6 as seen in FIG. 8. While, as seen in FIG. 8, a lower side forms the optical opening for the optical system of the digital microscope, the touch display is arranged in an upper area. It can also be said that the optical output is provided in a first, lower region of the robot head 6, while the touch display is provided at a second, upper region of the robot head, wherein the first and second regions form terminal regions facing away from each other. The touch display 12 is thus arranged in such a way that during a normal operation, in which the surgical microscope or the microscope head creates an image looking down on the patient from above, the laterally arranged touch display is particularly clearly visible and operable for a healthcare professional. The actuation buttons 22 are arranged below the touch display 12 as seen in FIG. 8, so that the touch display forms the uppermost element, so to speak. Seen along a longitudinal axis of the robot head 6, the following is therefore provided in this order in axial position: touch display 12—operating button-optical output. The robot arm 4 is connected to the side of the robot head 6 via the joint 28 and the three dashed rotational indications are intended to show that the robot head may be oriented around three axes or has three degrees of freedom of rotation.

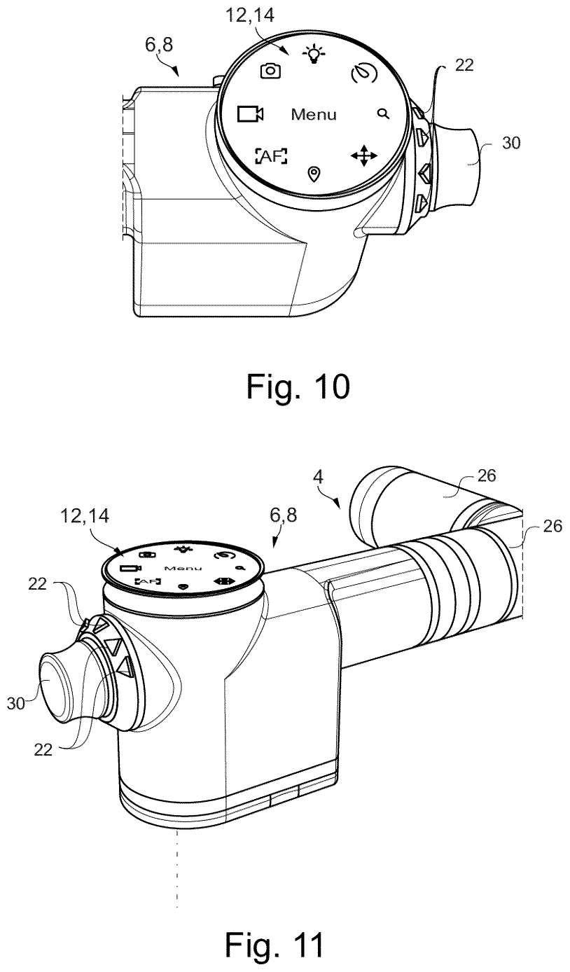

FIGS. 9 to 12 show a side view, a perspective top view and a perspective isometric view of a medical robot guiding system 1 according to a further preferred embodiment. The robot 2 is designed in the form of a robot-guided surgical microscope, the robot head 6 of which forms the end effector 8 as the microscope head and the position and orientation of the microscope head may be adjusted via the robot arm 4. The optical output is provided on a lower side as seen in FIG. 9, wherein the optical axis is shown as a dashed line. The touch display 12 is arranged on the side opposite the optical output. In this embodiment, the touch display 12 is circular in shape, wherein a center of the circular touch display 12 is substantially in extension of the optical axis, i.e. concentric to it, so to speak. It can also be said that the normal to the display is parallel to the optical axis, in particular on the optical axis (optical system and touch display are to a certain extent symmetrical to each other).

The touch display 12 forms an upper, flat surface at the front and is set off from the rest of the microscope head (and even from the robot arm 4) (i.e. forms a protrusion) in order to enable good operation. Seen in the direction of the longitudinal axis of the robot head or of the optical axis, from top to bottom, first the touch display 12 is provided, then an input means 30 in the form of a 3D mouse, such as a mouse manufactured by 3Dconnexion under the registered trademark SPACEMOUSE®, and then the optical output (with e.g. the last lens of the optical system). The input means 30 is also arranged in extension to a longitudinal axis of a cylindrical arm with a pivot joint 28 to a robot arm segment 26 on an opposite side of the connection to the robot arm 4. A zero axis of the 3D mouse is concentric to an axis of the robot arm segment 26.

Further actuation buttons 22 may also be used to control a movement and/or a zoom and/or other functions, depending on the function status in particular.

The robot head 6 is connected to the robot arm 4 (or to the robot arm segment 26) via a pivot joint as joint 28. An orthogonal to the touch display 12 is also perpendicular to an axis of rotation, wherein here the orthogonal to the touch display 12 is parallel to the optical axis.

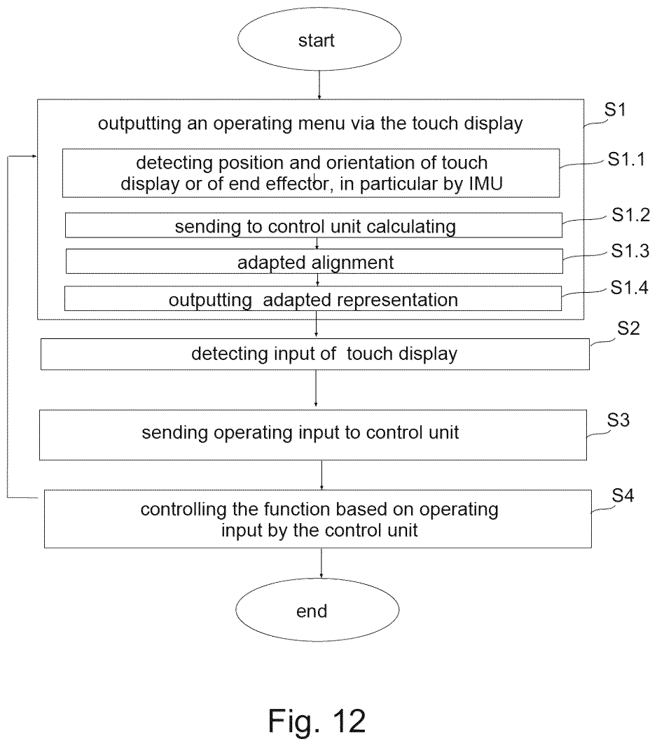

FIG. 12 shows a flowchart of an operating method according to a preferred embodiment.

In a first step S1, an operating menu 14 is output by the touch display 12.

Specifically, in this embodiment of the operating method, the output of the operating menu is solved by the following substeps. In a first substep S1.1, a position and an orientation (i.e. the pose) of the touch display 12 are detected (preferably also indirectly via the robot head 6), in particular by an inertial measurement unit (IMU) 16. Alternatively, the pose may also be detected via a detected (mechanical) robot kinematics or by a tracking camera/navigation camera of a navigation system.

The pose of the touch display 12 is then passed on to the control unit 12 in sub-step S1.2.

This then calculates an adapted alignment for the output in sub-step S1.3 and then outputs this adapted representation in sub-step S1.4 via the touch display 12.

In a subsequent second step S2, an input of the touch display 12 is then detected.

In a third step S3, the detected operating input is then sent to the control unit 12.

Finally, in step S4, a corresponding function as displayed and selected on the touch display 12 is controlled.

In particular, this may be a control of the robot 2.

### List of Reference Signs

1 medical robot guiding system
2 robot
4 robot arm
6 robot head
8 end effector
10 control unit
12 touch display
14 operating menu
15 external monitor
16 inertial measurement unit (IMU)
18 end effector-control unit
20 stop button
22 actuation button
24 robot base
26 robot-arm segment/robot arm link
28 joint
30 input means
P patient
S1 Step outputting an operating menu via the touch display
S1.1 Step detecting position and orientation by IMU
S1.2 Step sending to control unit
S1.3 Step calculating adapted alignment
S1.4 Step outputting the adapted representation
S2 Step detecting an input of the touch display
S3 Step sending operating input to control unit
S4 Step controlling a function

1-15. (canceled)

16. A medical robot guiding system for a surgical intervention on a patient, the medical robot guiding system comprising:
a robot with a robot arm and a robot head terminally connected to the robot arm, with an end effector on the robot head or as the robot head;
a control unit configured to control and move at least the robot;
at least one touch display configured to visually output at least one operating menu and to detect a touch-sensitive input as operating input and to send the operating input to the control unit;
the at least one touch display being rigidly fixed to the end effector and configured to dynamically move with the end effector.

17. The medical robot guiding system according to claim 16, wherein the medical robot guiding system comprises a surgical microscope with a microscope head connected to the robot arm as the robot head.

18. The medical robot guiding system according to claim 16, wherein the medical robot guiding system comprises a navigation system with a camera system connected to the robot arm.

19. The medical robot guiding system according to claim 16, wherein the medical robot guiding system is adapted to output a robot control menu as an operating menu via the at least one touch display in order to control the robot via an operating input.

20. The medical robot guiding system according to claim 16, wherein the medical robot guiding system is adapted to output a visualization control menu as an operating menu via the at least one touch display in order to control settings of a visualization system.

21. The medical robot guiding system according to claim 16, wherein the medical robot guiding system is adapted to switch between at least two different control menus in the at least one touch display, in order to control at least two different functions of the medical robot guiding system.

22. The medical robot guiding system according to claim 16, wherein the at least one touch display fixed to the robot head has a sterile sheathing in order to provide a sterile barrier with respect to a sterile intervention site.

23. The medical robot guiding system according to claim 16, further comprising an inertial measurement unit on or in the robot head, the inertial measurement unit configured to detect a position and/or orientation of the at least one touch display, wherein the medical robot guiding system is configured to adapt an alignment of a visual output of the at least one touch display based on the position and/or orientation of the at least one touch display.

24. The medical robot guiding system according to claim 16, wherein the at least one touch display has a radio communication module in order to establish a wireless data connection to the control unit.

25. The medical robot guiding system according to claim 16, wherein the at least one touch display has a display diagonal or a display diameter of at least 4 cm and/or a maximum of 20 cm.

26. The medical robot guiding system according to claim 16, wherein the robot head has at least one actuation button, and the at least one touch display is arranged directly adjacent to the at least one actuation button and is adapted to display a current functional status of the at least one actuation button.

27. The medical robot guiding system according to claim 16, further comprising a surgical microscope with a microscope head connected to the robot arm as the robot head, wherein an optical output forms a first side of the microscope head and opposite to the first side, the at least one touch display is arranged on a front side.

28. The medical robot guiding system according to claim 16, wherein:
the at least one touch display has a circular shape with a center point,
the robot is designed as a robot-guided surgical microscope, and
the center point lies in an extension of an optical axis.

29. A computer-readable storage medium comprising instructions which, when executed by a computer, cause the computer to execute method steps for controlling the medical robot guiding system according to claim 16.
